(19) United States
(12) Patent Application Publication
(10) Pub. No.: US 2014/0017223 A1
(43) Pub. Date: Jan. 16, 2014

(54) Pharmaceutical Compositions Comprising A Pancreatic Enzyme Preparation With Viral Infectivity Reduced Below A Significant Level And Methods Of Preparing And Using The Same

(71) Applicant: Aptalis Pharma Canada Inc., (US)

(72) Inventors: Peter TIJSSEN, Mont-Saint-Hilaire (CA); Jozsef SZELEI, Mont-Saint-Hilaire (CA)

(73) Assignee: Aptalis Pharma Canada Inc., Mont-Saint-Hilaire (CA)

(21) Appl. No.: 13/874,813

(22) Filed: May 1, 2013

Related U.S. Application Data
(63) Continuation of application No. 13/618,446, filed on Sep. 14, 2012, now abandoned.
Application No. 13/618,446 is a continuation of application No. PCT/IB2011/000580, filed on Mar. 18, 2011.
(60) Provisional application No. 61/315,813, filed on Mar. 19, 2010.

(51) Int. Cl. A61K 38/54

(52) U.S. Cl. CPC A61K 38/54
USPC 424/94.21

(57) ABSTRACT

The present invention provides for pharmaceutical compositions comprising pancreatic enzyme preparations (PEPs) with viral infectivity reduced below significant levels and having high enzymatic activity. The PEPs can comprise lipases, proteases, amylases, non-enveloped viruses (e.g., porcine parvovirus (PPV), porcine circovirus type 2 (PCV-2), porcine encephalomyocarditis virus (EMCV)), and enveloped viruses (e.g., vesicular stomatitis virus (VSV), and influenza A (IFA)). The present invention also includes methods of treating pancreatic insufficiency by administering these pharmaceutical compositions and methods of making the same by treating the PEP with beta-propiolactone (BPL) to reduce viral infectivity.

This application is a continuation of U.S. patent application Ser. No. 13/618,446, filed on Sep. 14, 2012, which is a continuation of International Patent Application No. PCT/IB2011/000580, filed Mar. 18, 2011, which claims the benefit of U.S. Provisional Application No. 61/315,813, filed Mar. 19, 2010, which is hereby incorporated by reference.

### FIELD OF THE INVENTION

The present invention relates to pancreatic enzyme preparations having reduced viral infectivity and in particular such preparations treated with beta-propiolactone (BPL), pharmaceutical compositions containing them, and methods of preparing them.

### BACKGROUND OF THE INVENTION

Pancreatic exocrine insufficiency is a major consequence of pancreatic diseases (e.g., chronic pancreatitis, cystic fibrosis, severe acute necrotizing pancreatitis, and pancreatic cancer), extrapancreatic diseases such as celiac disease and Crohn's disease, and gastrointestinal and pancreatic surgical resection. Replacement of pancreatic exocrine function is important to avoid malnutrition-related morbidity and mortality. One therapy for pancreatic exocrine insufficiency is the oral administration of pancreatic enzymes to provide the duodenal lumen with sufficient active lipase at the time of gastric emptying of nutrients.

Pancreatic enzyme preparations (PEPs) obtained from animal sources have been used in various forms over the past seventy years to partially remedy enzyme deficiency in patients suffering from various pancreatic enzyme deficiency and digestive disorders. PEPs typically contain a combination of at least three categories of enzymes including: lipases, proteases, and amylases, which are important in the digestion of fats, proteins and sugars. One PEP known as pancrelipase is commercially available in the form of enteric coated particles incorporated into capsules which contain up to 35,000 USP units/capsule of pancrelipase (e.g., PANCRECARB® (Digestive Care, Inc.), ULTRASE® (Axcan Scandipharm Inc.), PANCREAZE™ (McNeil Pharmaceutical), COTAZYME® (Organon USA, Inc.), ZENPEP® (Eurand Pharmaceuticals) and CREON® (Solvay Pharmaceuticals, Inc.)). Since these enzymes are isolated from animal sources, they are susceptible to being contaminated with viruses, such as non-enveloped viruses (e.g., porcine parvovirus (PPV), porcine circovirus type 2 (PCV-2), porcine encephalomyocarditis virus (EMCV)) and enveloped viruses (e.g., vesicular stomatitis virus (VSV), and influenza A (IFA)) for material derived from swine. See the information from the FDA Antiviral Drugs Advisory Committee Meeting held in December 2008 available on the WorldWideWeb at fda.gov/ohrms/dockets/ac/cder08.html#AntiviralDrugs.

It has been reported that certain viruses can be inactivated using chemicals or other techniques. See, e.g., Mayr, Development of non-immunizing, paraspecific vaccine from attenuated pox viruses: a new type of vaccine, Berl Munch Tierarztl Wochenschr. 2001, 114:184-7; Argüello Villares, Viral haemorrhagic disease of rabbits: vaccination and immune response, Rev Sci Tech. 1991, 10:459-80; Horowitz, Inactivation of viruses found with plasma proteins, Biotechnology 1991, 19:417-30; Epstein and Fricke, Current safety of clotting factor concentrates, Arch Pathol Lab Med. 1990, 114:335-40; Stephan, Inactivation of hepatitis viruses and HIV in plasma and plasma derivatives by treatment with beta-propiolactone/UV irradiation, Curr Stud Hematol Blood Transfus. 1989, 56:122-7; Stephan, Virus safety of labile plasma products from the German viewpoint, Beitr Infusionsther. 1989, 24:40-5; Anderson et al., The role of specific IgE and beta-propiolactone in reactions resulting from booster doses of human diploid cell rabies vaccine, J Allergy Clin Immunol. 1987, 80:861-8; Prince et al., Beta-propiolactone/ultraviolet irradiation: a review of its effectiveness for inactivation of viruses in blood derivatives, Rev Infect Dis. 1983, 5:92-107; U.S. Publication Number 2006/0115376.

Currently, there is a need for virus-free or reduced-viral infectivity oral PEPs containing doses of active enzymes supplements.

### BRIEF SUMMARY OF THE INVENTION

The present inventors discovered that viruses in a pancreatic enzyme preparation (PEP) can be efficiently inactivated without eliminating the enzymatic activity of the PEP by treating the PEP with a chemical reagent—beta-propiolactone (BPL). Without being bound by any theory, the inactivation of viruses by BPL is based on the alkylation of the virus genome. The present inventors also discovered that PEP components other than the viruses, such as the pancreatic enzymes or nucleic acids that could potentially reduce the alkylation activity of BPL, do not interfere with the inactivation of the viruses in a PEP.

One embodiment of the present invention is a PEP having reduced viral infectivity. The preparation includes (i) one or more pancreatic enzymes treated with BPL and (ii) 3-hydroxypropionic acid (i.e., the hydrolysis product of BPL), BPL, or a mixture thereof. Preferably, the PEP has a PPV viral infectivity of less than about 10³ FFID₅₀/g PEP, as measured by the PPV FFID-infectivity assay described in U.S. Publication No. 2009/0226414, which is hereby incorporated by reference. Preferably, the PEP has a viral infectivity of at least 1 log below that of a similar preparation not treated with BPL, where the viral infectivity is that of non-enveloped viruses, enveloped viruses, PPV, EMCV, or a combination thereof.

Another embodiment is a pharmaceutical composition having reduced viral infectivity comprising (i) one or more pancreatic enzymes (or a PEP) treated with BPL and (ii) 3-hydroxypropionic acid, BPL, or a mixture thereof. Yet another embodiment is a method for treating pancreatic insufficiency in a patient in need thereof by administering to the patient a therapeutically effective amount of the PEP or pharmaceutical composition of the present invention.

Yet another embodiment is a method of preparing a PEP comprising the step of (a) reacting BPL with a preparation containing one or more pancreatic enzymes for a sufficient time to reduce the viral infectivity in the preparation. In one embodiment, the reaction in step (a) is performed by (i) adding BPL to a solution or suspension containing a preparation of one or more pancreatic enzymes, and (ii) incubating BPL in the solution in step (a) for a time sufficient to decrease the viral infectivity in the solution. The method can further include the step of (b) inactivating the BPL after the reaction in step (a). The BPL can be inactivated by incubation in an aqueous solution, by adding an inactivating agent (such as sodium thiosulfate), or by freezing the solution formed in step (a) and then lyophilizing it.

The pancreatic enzymes in any embodiments of the present invention can be formed recombinantly or derived from an animal source (hog, sheep and bovine). Suitable pancreatic enzymes include, but are not limited to, lipases, proteases, amylases, and any combination of any of the foregoing. A preferred mixture of pancreatic enzymes is pancrelipase of porcine origin.

### DETAILED DESCRIPTION OF THE INVENTION

### Definitions

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J., and Animal Cell Culture (Freshney, ed.: 1986).

The term “patient” refers to a human being or other animal.

Common abbreviations correspond to units of measure, techniques, properties or compounds as follows: “min” means minutes, “h” or “hr” means hour(s), “μL” or “μl” means microliter(s), “mL” or “ml” means milliliter(s), “mM” means millimolar, “M” means molar, and “mmole” means millimole(s).

The term “USP unit” refers to a unit used to measure the potency of a vitamin or drug, that is, its expected biological effects. For each substance to which this unit applies, the U.S. Food and Drug Administration has determined the biological effect associated with a dose of 1 USP unit. Other quantities of the substance can then be expressed in terms of this standard unit. In most cases, the USP unit is equal to the international unit (IU).

One USP Unit of amylase activity is contained in the amount of pancrelipase that decomposes starch at an initial rate such that 0.16 μEq of glycosidic linkage is hydrolyzed per minute under the conditions of the Assay for amylase activity from the Official Monograph for Pancrelipase (The 2009 United States Pharmacopia 32/National Formulary 27) incorporated herein by reference.

One USP Unit of lipase activity is contained in the amount of pancrelipase that liberates 1.0 μEq of acid per minute at pH 9.0 and 37° C. under the conditions of the Assay for lipase activity from the Official Monograph for Pancrelipase (The 2009 United States Pharmacopia 32/National Formulary 27) incorporated herein by reference.

One USP Unit of protease activity is contained in the amount of pancrelipase that under the conditions of the Assay for protease activity from the Official Monograph for Pancrelipase (The 2009 United States Pharmacopia 32/National Formulary 27) incorporated herein by reference, hydrolyzes casein at an initial rate such that there is liberated per minute an amount of peptides not precipitated by trichloroacetic acid that gives the same absorbance at 280 nm as 15 nmol of tyrosine.

### Pancreatic Enzyme Preparations (PEPs)

Suitable pancreatic enzyme preparations (PEPs) may contain a mixture of digestive enzymes such as lipases, proteases, and amylases. PEPs may be derived from animal sources. Animal derived PEPs can be contaminated with at least one or more viruses, such as PPV, PCV-2, EMCV, VSV, and influenza A, and nucleic acids. PEPs can also contain residual amounts of isopropanol (IPA).

Active enzymes which may be present in PEPs include, but are not limited to, pancreatic enzymes, such as pancrelipase (a mixture of lipases, proteases, and amylases) or pancreatin. Other active enzymes which may be present in a PEP include: (i) active proteases including, but not limited to: trypsin, E.C. (Enzyme Commission Number) 3.4.4.4; chymotrypsin, E.C. 3,4,4,5; chymotrypsin B, E.C. 3,4,5,6; pancreatopeptidase E, E.C. 3.4.4.7; carboxypeptidase A, E.C. 3.4.2.1; and carboxypeptidase B, E.C. 3.4.2.2; (ii) active lipases, including, but not limited to: glycerol ester hydrolase (Lipase), E.C. 3.1.1.3; phospholipase A₂, E.C. 3.1.1.4; and sterol ester hydrolase, E.C. 3.1.1.13; (iii) nucleases, such as, but not limited to: ribonuclease, E.C. 2.7.7.16 and deoxyribonuclease, E.C. 3.1.4.5; and (iv) active amylases such as α-Amylase, E.C. 3.2.1.1.

In one preferred embodiment, the PEP includes a mixture of pancreatic enzymes, including proteases, amylases, and lipases, and optionally nucleases such as ribonuclease. The enzymes may be derived from animal sources such as hog, sheep and bovine. Co-lipase may also be included in the PEP. It should be understood that, as used herein, the term “enzyme” includes not only the already activated form but also the zymogen precursor which is capable of being transformed into the active form in mammalian intestinal fluid.

In one embodiment, the PEP is pancrelipase having a lipase activity of from about 69 to about 120 U USP/mg, amylase activity of greater than or equal to about 216 U USP/mg, protease activity of greater than or equal to about 264 U USP/mg, and total protease activity of greater than or equal to about 264 U USP/mg.

Lipase activities in the PEP (before or after BPL treatment) can range from about 1,000 to about 150,000 International Units (U). Amylase activities in the PEP (before or after BPL treatment) can range from about 3,000 to about 500,000 U. Proteases activities in the PEP (before or after BPL treatment) can range from about 3,000 to about 500,000 U. In another embodiment, the PEP comprises from about 2,000 to about 75,000 USP units of lipase, from about 8,000 to about 250,000 U proteases, and from about 8,000 to about 250,000 U amylases. In yet another embodiment, the PEP comprises from about 2,000 to about 40,000 USP units of lipase, from about 8,000 to about 160,000 U proteases, and from about 8,000 to about 160,000 U amylases.

Lipase activities in the PEP (before or after BPL treatment) can be from about 3000 to about 25,000 IU, from about 4500 to about 25,000 IU, for example from about 4500 to about 5500 IU, from about 9000 to about 11,000 IU, from about 13,500 to about 16,500 IU, and from about 18,000 to about 22,000 IU. Amylase activities in the PEP (before or after BPL treatment) can be from about 8100 to about 180,000 IU, for example from about 8000 to about 45,000 IU, from about 17,000 to about 90,000 IU, from about 26,000 to about 135,000 IU, from about 35,000 to about 180,000 IU. Protease activities in the PEP (before or after BPL treatment) can be from about 8000 to about 134,000 IU, for example from about 8000 to about 34,000 IU, from about 17,000 to about 67,000 IU, from about 26,000 to about 100,000 IU, from about 35,000 to about 134,000 IU. In one embodiment, the lipase activity ranges from about 4500 to about 5500 IU, the amylase activity ranges from about 8000 to about 45,000 IU, and the protease activity ranges from about 8000 to about 34,000 IU. In another embodiment, the lipase activity ranges from about 9000 to about 11,000 IU, the amylase activity ranges from about 17,000 to about 90,000 IU, and the protease activity ranges from about 17,000 to about 67,000 IU. In yet another embodiment, the lipase activity ranges from about 13,500 to about 16,500 IU, the amylase activity ranges from about 26,000 to about 135,000 IU, and the protease activity ranges from about 26,000 to about 100,000 IU. In still another embodiment, the lipase activity ranges from about 18,000 to about 22,000 IU, the amylase activity ranges from about 35,000 to about 180,000 IU, and the protease activity ranges from about 35,000 to about 134,000 IU. In still another embodiment, the lipase activity can be about 5,000 or about 30,000 lipase PhEur.

The ratio of amylase/lipase (in USP units) in the PEP (before or after BPL treatment) can range from about 1.8 to about 8.2, for example from about 1.9 to about 8.2, and about 2.0 to about 8.2. The ratio of protease/lipase in the PEP (before or after BPL treatment) can range from about 1.8 to about 6.2, for example about 1.9 to about 6.1, and about 2.0 to about 6.1.

In one embodiment, the ratio of amylase:lipase in the PEP (before or after BPL treatment) can be in the range of from about 1 to about 10, for example from about 2.38 to about 8.75 (where the amylase assay is performed according to USP). The ratios of protease:lipase in the PEP (before or after BPL treatment) can be in the range of from about 1.00 to about 8.00, for example from about 1.86 to about 5.13 (where the protease assay is performed according to USP).

In one embodiment, the PEP contains lipases, proteases, and amylases where (i) the ratio of amylase to lipase in the PEP ranges from about 3:1 to about 6:1 and (ii) the ratio of protease to lipase ranges from about 3:1 to about 6:1 (as measured by USP units).

The table below provides additional suitable pancreatic enzyme mixtures, where the amount of lipases, proteases, and amylases are provided in USP units.

| | Mixture D | | Mixture E | | Mixture F | | Mixture G | |
|---|---|---|---|---|---|---|---|---|
| | Activity | | Activity | | Activity | | Activity | |
| | (USP units) | | (USP units) | | (USP units) | | (USP units) | |
| Enzyme category | min | max | min | max | min | max | min | max |
| Lipases | about | about | about | about | about | about | about | about |
| | 2500 | 6500 | 12500 | 17500 | 19 500 | 45 000 | 1000 | 60 000 |
| Proteases | about | about | about | About | about | about | about | about |
| | 10 000 | 35000 | 30 000 | 80 000 | 50 000 | 160 000 | 3000 | 360 000 |
| Amylases | about | about | about | About | about | about | about | about |
| | 10 000 | 35000 | 30 000 | 80 000 | 50 000 | 160 000 | 3000 | 360 000 |

In another embodiment, the activities of lipase, protease, and amylase can be those described in Tables A and B, below:

TABLE A

| | | | | | Ratio | |
|---|---|---|---|---|---|---|
| | | Activity (IU) | | | Amylase/ | Protease/ |
| Formulation | | Lipase | Amylase | Protease | Lipase | Lipase |
| 1 | Min | about | about 8100 | about | about 1.8 | about 1.8 |
| | | 4500 | | 8100 | | |
| | Max | about | about | about | about 8.2 | about 6.2 |
| | | 5500 | 45000 | 34000 | | |
| 2 | Min | about | about | about | about 1.9 | about 1.9 |
| | | 9000 | 17100 | 17100 | | |
| | Max | about | about | about | about 8.2 | about 6.1 |
| | | 11000 | 90000 | 67000 | | |
| 3 | Min | about | about | about | about 1.9 | about 1.9 |
| | | 13500 | 26100 | 26100 | | |
| | Max | about | about | about | about 8.2 | about 6.1 |
| | | 16500 | 135000 | 100000 | | |
| 4 | Min | about | about | about | about 2.0 | about 2.0 |
| | | 18000 | 35100 | 35100 | | |
| | Max | about | about | about | about 8.2 | about 6.1 |
| | | 22000 | 180000 | 134000 | | |
| 5 | Min | about | about 6800 | about | about 1.8 | about 1.8 |
| | | 3800 | | 6800 | | |
| | Max | about | about | about | about 8.2 | about 6.2 |
| | | 4600 | 37700 | 28500 | | |
| 6 | Min | about | about | about | about 1.8 | about 1.8 |
| | | 9500 | 17100 | 17100 | | |
| | Max | about | about | about | about 8.2 | about 6.2 |
| | | 11500 | 94300 | 71300 | | |
| 7 | Min | about | about | about | about 1.8 | about 1.8 |
| | | 15100 | 27200 | 27200 | | |
| | Max | about | about | about | about 8.2 | about 6.2 |
| | | 18500 | 151700 | 114700 | | |
| 8 | Min | about | about | about | about 1.8 | about 1.8 |
| | | 18900 | 34000 | 34000 | | |
| | Max | about | about | about | about 8.2 | about 6.2 |
| | | 23100 | 189400 | 143200 | | |
| 9 | Min | about | about 9700 | about | about 1.8 | about 1.8 |
| | | 5400 | | 9700 | | |
| | Max | about | about | about | about 8.2 | about 6.2 |
| | | 6600 | 54100 | 40900 | | |
| 10 | Min | about | about | about | about 1.8 | about 1.8 |
| | | 10800 | 19400 | 19400 | | |
| | Max | about | about | about | about 8.2 | about 6.2 |
| | | 13200 | 108200 | 81800 | | |
| 11 | Min | about | about | about | about 1.8 | about 1.8 |
| | | 21600 | 38900 | 38900 | | |
| | Max | about | about | about | about 8.2 | about 6.2 |
| | | 26400 | 216500 | 163700 | | |

TABLE B

| | | | | | Ratio | |
|---|---|---|---|---|---|---|
| | | Activity (PhEur) | | | Amylase/ | Protease/ |
| Formulation | | Lipase | Amylase | Protease | Lipase | Lipase |
| 12 | Min | about | about 3900 | about 110 | about 0.43 | about |
| | | 9000 | | | | 0.012 |
| | Max | about | about | about | about 1.98 | about |
| | | 11000 | 21700 | 2150 | | 0.196 |
| 13 | Min | about | about 9800 | about 280 | about 0.43 | about |
| | | 22500 | | | | 0.012 |
| | Max | about | about | about | about 1.98 | about |
| | | 27500 | 54300 | 5400 | | 0.196 |
| 14 | Min | about | about | about 450 | about 0.43 | about |
| | | 36000 | 15600 | | | 0.012 |
| | Max | about | about | about | about 1.98 | about |
| | | 44000 | 86900 | 8600 | | 0.196 |

Below is a table for converting units of amylase, lipase, and protease.

Conversion values for units of enzyme activity

| | | | | |
|---|---|---|---|---|
| Amylase | 1 PhEur | equals 1 FIP | equals 1 BP | equals 4.15 USP units |
| | unit | unit | unit | |
| Lipase | 1 PhEur | equals 1 FIP | equals 1 BP | equals 1 USP unit |
| | unit | unit | unit | |
| Protease | 1 PhEur | equals 1 FIP | equals 1 BP | equals 62.5 USP units |
| | unit | unit | unit* | |

*Only free protease for pancreatin; total protease for pancreatic extract.
BP—British Pharmacopoeia;
FIP—Federation Internationale Pharmaceutique;
PhEur—European Pharmacopoeia.

The enzymes may be in concentrated form. The enzymes may be in an amorphous powder or crystalline form. A PEP solution may be filtered during manufacturing. Lipase activity in a PEP after BPL treatment is typically not less than about 24 USP Units/mg PEP, and more preferably not less than about 39 Units/mg PEP or not less than about 75 USP Units/mg PEP. In one embodiment, the lipase activity in a PEP after BPL treatment is from about 86 to about 120 Units/mg PEP. Protease activity in a PEP after BPL treatment is typically not less than about 100 Units/mg PEP and preferably not less than about 200 Units/mg PEP or not less than about 240 USP Units/mg PEP. In one embodiment, the protease activity in a PEP after BPL treatment is about 280 to about 440 USP Units/mg PEP. Amylase activity in a PEP after BPL treatment is typically not less than about 100 Units/mg PEP and preferably not less than about 200 USP Units/mg PEP. In one embodiment, the amylase activity in a PEP after BPL treatment is about 210 to about 570 USP Units/mg PEP.

Infectious virus particles or an infectious virus found in PEPs can be of any type including those found in porcine sources, such as PPV. PPV is a non-enveloped, small DNA virus (Bergeron et al., Virology, 1993, 197(1):86-98; and J. Virol. 1996 April; 70(4): 2508-2515; Simpson et al., J. Mol. Biol., 2002 315(5):1189-98; Szelei et al., 2006. Porcine parvovirus pp. 434-445, in: Parvoviruses (Kerr et al., eds), Hodder Arnold Publ., London, UK) and is the most prominent among the viruses found in PEPs. PPV has a high degree of stability in the environment, and during manufacturing it is resistant to hydrolytic enzymes and relatively high temperature, and remains infective throughout a wide pH range. Since PPV is highly resistant, it can be a model virus for other viruses that can be found in PEPs.

Other non-enveloped viruses that could be found in PEPs include EMCV (porcine encephalomyocarditis virus, also known as MEV), swine hepatitis virus (including HEV (swine hepatitis E virus)), SVDV (swine vesicular disease virus, also known as PEV9), vesicular exanthema virus, porcine circovirus (including PCV1 (porcine circovirus 1) and PCV2 (porcine circovirus 2)), porcine rotavirus (Rota A), reovirus (including reovirus type 3 which is known as Reo3), foot and mouth disease virus, porcine teschovirus 1 (PTV1, also known as PEV1), porcine adenovirus, and porcine respiratory coronavirus. Enveloped viruses, such as pseudorabiesvirus, VSV (vesicular stomatitis virus), IFA (influenza A), rabies virus, African swine fever virus, transmissible gastroenteritis virus, classical swine fever virus, West Nile virus, suipoxvirus, hantavirus, porcine cytomegalovirus, porcine lymphotropic herpesvirus, porcine endogenous retrovirus, porcine respiratory reproductive syndrome virus, paramyxovirus, and encephalomyelitis virus could also be found in PEPs. See the information from the FDA Antiviral Drugs Advisory Committee Meeting held in December 2008 available on the WorldWideWeb at fda.gov/ohrms/dockets/ac/cder08.html#AntiviralDrugs. PEPs could also potentially contain emerging viruses such as emerging enveloped or non-enveloped adventitious agents (e.g., ebola virus) or mutant viruses. Other viruses which can be found in PEPs include pseudorabies virus, bovine viral diarrhea virus, picornaviridae (including porcine picornaviridae), reoviridae (including porcine reoviridae), astroviridae (including porcine astroviridae), adenoviridae (including porcine adenoviridae) and hepeviridae (including porcine hepeviridae).

In one embodiment, the allowable level of infectious PPV viruses is below about 10⁵ FFID₅₀/g PEP (corresponding to about 10^{4.5 TCID}₅₀), preferably below about 10³ FFID₅₀/g PEP, and most preferably below about 10² FFID₅₀/g PEP as measured by the PPV FFID-infectivity assay described in U.S. Publication No. 2009/0226414. In another embodiment, the allowable level of another infectious virus, such as EMCV, HEV, SVDV, PCV1, PCV2, VSV, and IFA, is below the detection limit of a publicly available assay technique (e.g., less than about 10² infectious virus particles/g PEP), and preferably about 0 infectious virus particles/g PEP. The levels of virus particles can be measured before, after, or both before and after the treatment of PEPs using BPL as discussed below.

In addition, the BPL may act as an antimicrobial agent (e.g., as an antibacterial agent).

The PEP may be incorporated or formed into a pharmaceutical composition, e.g., in admixture with a suitable pharmaceutical excipient, diluent and/or carrier selected with regard to the intended route of administration and standard pharmaceutical practice. The compositions of the invention can be formulated for administration in any convenient way for use in a human. The PEP can be administered as an oral dosage form. Oral dosage forms include powders, tablets, mini-tablets, micro-tablets, uncoated dosage forms, coated dosage forms, microspheres, prills, caplets, gelcaps, capsules, and medical foods. Tablets, for example, can be made by compression techniques known in the art. Typically, these enzymes are orally administered in the form of enteric-coated mini-microspheres to avoid acid-mediated lipase inactivation and to ensure gastric emptying of enzymes in parallel with nutrients.

Suitable pharmaceutically acceptable excipients include, but are not limited to, diluents, binding agents, lubricants, glidants, disintegrants, and coloring agents. Other components such as preservatives, stabilizers, dyes and flavoring agents may be included in the dosage form. Examples of preservatives include sodium benzoate, ascorbic acid and esters of p-hydroxybenzoic acid. Antioxidants and suspending agents may be also included. Pharmaceutically acceptable excipients, diluents, and carriers for therapeutic use are known in the pharmaceutical art, and are described, for example, in Remington: The Science and Practice of Pharmacy, Lippincott Williams & Wilkins (A. R. Gennaro edit. 2005).

### Beta-Propiolactone (BPL)

Beta-propiolactone (BPL, 2-oxetanone) is a small organic compound of the lactone family with a four-membered ring. It is a clear, colorless liquid with a slightly sweet odor and is highly soluble in water. Because of its high water-solubility and small size, BPL easily diffuses in solutions with high viscosity and multiparticulate matter, such as solutions with high protein content. Beta-propiolactone slowly reacts with water and hydrolyzes to produce 3-hydroxypropionic acid (hydracryclic acid), a non-toxic compound. Thus, BPL activity is self-limiting and there are no residual toxic contaminants.

According to one embodiment, BPL is added to an aqueous or organic solution or suspension containing a PEP in an amount effective to reduce viral infectivity to a level that is acceptable for administration of the PEP to a patient. For instance, in one embodiment, to a solution containing from about 100 to about 200 mg PEP/ml, from about 0.004% to about 1.0% (v/v) of BPL is added. In preferred embodiments, from about 0.1 to about 0.8% (v/v) BPL is added to a solution containing from about 100 to about 200 mg PEP/ml or from about 0.2 to about 0.4% (v/v) BPL is added to a solution containing from about 100 to about 200 mg PEP/ml. Without being bound by any theory, the inventors theorize that BPL inactivates viruses by alkylating them. Thus, a PEP having reduced viral infectivity after treatment with BPL can contain inactivated viruses.

The BPL-containing PEP can be incubated for about 30 minutes to about 48 or about 72 hours (or longer). According to one embodiment, the BPL-containing PEP is incubated for about 24 to about 72 hours, or for about 48 hours at, e.g., from about 5 to about 50° C., from about 15 to about 40° C., or room temperature or about 25° C. In another embodiment, the BPL-containing PEP is incubated for from about 15 minutes to about 2 hours (e.g., for about 30 minutes) at, e.g., from about 15 to about 40° C. or room temperature or about 25° C. In another embodiment, the BPL-containing PEP is incubated for from about 15 minutes to about 12 hours (e.g., for about 4 hours) at about 4° C.

After incubation, the BPL can be inactivated and/or removed from the PEP. BPL can be inactivated by incubation in aqueous media to form non-toxic 3-hydroxypropionic acid. The maximum half-life of BPL in water is approximately 210 minutes or less at 25° C. (Hoffman and Warshowsky, Beta-Propiolactone Vapor as a Disinfectant, Appl Microbiol 1958, 6:358-362. The rate of BPL inactivation depends on environmental factors such as pH, temperature and ionic strength of the buffer (Budowsky and Zalesskaya, Principles of selective inactivation of viral genome (V) Rational selection of conditions for inactivation of the viral suspension infectivity to a given extent by the action of beta-propiolactone, Vaccine 1991, 9:319-325). BPL is consumed by hydrolysis and by the alkylation reaction targeting molecules for inactivation. Thus, after eight hours of incubation at 20° C., the level of BPL could be reduced at least eight times.

A PEP having reduced viral infectivity after treatment with BPL containing (i) one or more pancreatic enzymes and (ii) 3-hydroxypropionic acid, BPL, or a mixture thereof can have a viral infectivity of at least about 1 log below, of at least about 2 logs below, of at least about 3 logs below, of about 1 log below, of about 2 logs below, or of about 3 logs below that of a preparation not treated with BPL as measured by one of the methods for measuring viral infectivity activity discussed below such as the PPV FFID-infectivity assay described in U.S. Publication No. 2009/0226414. The relevant viral infectivity of the PEP can be that of non-enveloped viruses, enveloped viruses, all viruses, or a particular virus (e.g., PPV or EMCV) in the PEP.

One method for rapid inactivation of the BPL is by adding a BPL inactivating agent, such as sodium thiosulfate. A final concentration of 100 mM sodium thiosulfate in the Examples below was more than sufficient to deactivate the BPL to achieve completion within minutes at 4° C. In one embodiment, the minimum concentration of sodium thiosulfate used for the neutralization of BPL is in about a 1.7-fold molar excess (e.g., a 0.4% BPL (v/v) (63.61 mM) solution would be mixed with at least one-tenth of the volume of 1.08 M sodium thiosulfate solution).

The BPL-containing PEPs can also be frozen and lyophilized to inactivate the BPL. Samples can be fast frozen to stop the inactivation reaction of PPV with the BPL. During the storage of the frozen material, BPL can slowly degrade in the presence of ice and partially evaporate. Additional PPV inactivation can also occur slowly under these conditions. For example, the preparations can be placed in a mixture of acetone and dry ice for fast freezing followed by storage at −80° C. and then lyophilized at −45° C. for about two days. The length of lyophilization in the experiment depends on the volume of the sample; typically, smaller volumes require less time.

### Measuring Viral Infectivity Activity

The activity of viruses in the initial and treated PEP can be performed by any method known in the art. Preferably, the viral infectivity of one or more viruses is measured in the treated PEP. In one embodiment, the viral infectivity of PPV, EMCV, PCV-2 and/or other viruses are measured.

In one embodiment, the infectivity assay uses cell cultures growing in tissue culture wells to which samples are applied and infection of the cells is observed.

For example, two preparations can be tested for PPV or another virus on one 96-well plate, with the positive controls incubated on another parallel plate. For PPV the samples can be serially diluted (for example, 1:4 in each step for a total of 8 dilutions) in cell culture medium prior to application and incubated with cell cultures for at least 5 days at 37° C. in 5% CO₂. After sample removal and the fixation of the cells by paraformaldehyde, the presence of PPV (or other) virus infection is monitored in each well by an anti-PPV antibody using a standard immunofluorescent assay. The number of wells that contain positively stained cells are recorded in a table. Fluorescence Focus Infectious Doses (FFID₅₀) are calculated according to the Karber formula:

FFID₅₀=10^{(D-0.5d+d(S))};

where D=log₁₀ of the highest dilution demonstrating 100% infection based on the presence of at least one infectious fluorescent focus in every well of all replicates at the given dilution, d=log₁₀ of the dilution factor and S=the ratio of the total number of wells with infection to the number of wells per dilution. All data are recalculated for 1-gram dry PEP, e.g., pancrelipase, sample after the consideration of processed sample volumes and dilutions. In certain embodiments, the assay is repeated several times using six parallel wells to quantify the level of infectious viruses.

Other assays to measure viral infectivity include the TCID₅₀ assay as discussed in Hierholzer, J. C., Killington, R. A., 1996. Virus isolation and quantitation. In: Mahy, B. W. J., Kangro, H. O. (Eds.), Virology methods manual. Academic Press, London, pp. 25-46.

The activity of the enzymes of a PEP can be toxic to the cells used for detecting viral infectivity and the enzymes may also inhibit viral infection of the test cell lines in other ways. Other compounds present in the preparations may also interfere with viral, e.g., PPV, infection. Since cell proliferation is necessary for PPV replication, the presence of any of these cytostatic or toxic enzymes can further reduce the efficiency of viral infection and prevent an accurate determination of viral infectivity in PEP samples. Therefore, it can be useful to treat PEP samples prior to determining PPV activity. After the treatment, for example as described below, the samples can be dissolved in cell culture medium in a volume that is equal to or less than the original sample volume to preserve the sensitivity of the infectivity assay.

U.S. Patent Publication No. 2009/0226414, incorporated by reference herein, provides a reproducible and efficient method for detecting an infectious non-enveloped virus, such as PPV, in an enzyme preparation (e.g., a pancreatic enzyme preparation) by treating the preparation prior to determining its viral infectivity. This method preserves the infectious non-enveloped virus while substantially eliminating the toxic enzyme materials from the PEP samples.

The steps of this method include:

(a) extracting a sample of the preparation at least two times with chloroform to produce a clarified sample with an upper phase and a lower phase;

(b) precipitating an aliquot of the upper phase from step (a) with polyethylene glycol (PEG);

(c) suspending the product from step (b) in a buffer;

(d) precipitating the product from step (c) with PEG;

(e) suspending the product from step (d) in a solution;

(f) removing excess PEG and fine particles from product (e) by chloroform, this allows (i) in step (e) the suspending in a 10-times smaller volume to increase the overall sensitivity up to 10-fold; and (ii) removal of the remaining interfering materials from the least diluted steps of the most toxic samples; and

(g) detecting the presence of or measuring the amount of infectious virus in the solution.

Heat inactivation, prolonged storage, ultracentrifugation, and other extraction methods can also be useful to treat samples prior to infectious virus measurement under certain circumstances.

The level of inactivation for other viruses, such as PCV-2 and EMCV, can be measured using a TCID₅₀ assay (Hierholzer, J. C., Killington, R. A., 1996. Virus isolation and quantitation. In: Mahy, B. W. J., Kangro, H. O. (Eds.), Virology methods manual. Academic Press, London, pp. 25-46).

### Measuring Enzymatic Activity

Enzymatic activity for the enzymes of a PEP (e.g., proteases, lipases, and amylases) can be determined, for example, by using the United States Pharmacopeia (USP) monographed methods (The United States Pharmacopeia and the National Formulary (USP 31/NF 26). 2008. Rockville (MD): United States Pharmacopeial Convention, Inc.).

Methods of Treatment with the PEP

The PEP or a pharmaceutical composition containing the PEP with a reduced viral infectivity can be administered in an effective amount to control steatorrhea in a patient or to treat a patient with partial or complete exocrine pancreatic insufficiency. The pancreatic insufficiency can be that caused by cystic fibrosis (CF), chronic pancreatitis due to alcohol use or other causes, severe acute necrotizing pancreatitis, pancreatic cancer, surgery (pancreatico-duodenectomy or Whipple's procedure, with or without Wirsung duct injection, total pancreatectomy), obstruction (pancreatic and biliary duct lithiasis, pancreatic and duodenal neoplasms, ductal stenosis), other pancreatic disease (e.g., hereditary, post traumatic and allograft pancreatitis, hemochromatosis, Shwachman's Syndrome, lipomatosis, and hyperparathyroidism), extrapancreatic diseases such as celiac disease and Crohn's disease, poor mixing (Billroth II gastrectomy, other types of gastric bypass surgery, gastrinoma), and diabetes type I and II.

A “therapeutically effective amount” generally refers to the amount of a compound or composition that, when administered to a patient (such as a human) for treating a state, disorder or condition, is sufficient to effect such treatment. The “therapeutically effective amount” will vary depending on the compound or composition, the disease and its severity and the age, weight, physical condition and responsiveness of the animal to be treated.

### EXAMPLES

Two sets of experiments were performed to study PPV and EMCV inactivation using BPL. Examples 1-7 show that BPL inactivates PPV and EMCV in PEP preparations. The second set of Examples (Examples 8-11) determined the degree of virus inactivation in PEP preparations after 30 min of incubation with BPL at 25° C. and subsequent inactivation of the remaining BPL by sodium thiosulfate or by freezing followed by lyophilization. The enzymatic activities of the BPL-treated PEPs were also determined for the samples described in Examples 8-11.

### Example 1

### Inactivation of PPV by BPL with Conc. Between 0.1-0.8% (v/v) in PEP Preparations

### The PPV was Titered Using a Dilution Method

Pancrelipase powders from 2 different lots were each dissolved in 100 mM Tris, pH=8.3, at a concentration of 100 mg/ml. Two ml non-purified PPV viral stock (prepared using standard protocols (Arella et al., Physicichemical properties, production, and purification of parvoviruses. 1990, In Tijssen, P. (ed.), CRC handbook of parvoviruses. Boca Raton, Fla.: CRC Press, 11-30)) were added to 38 ml of the pancrelipase solution. The virus was mixed with the PEP using slow shaking at room temperature for one day. Aliquots were taken from the PPV-spiked pancrelipase stocks and each was incubated with different concentrations of BPL (available from Sigma-Aldrich, St. Louis, Mo.) (except the control) for 48 hours at room temperature (20° C.). The level of viral inactivation was measured by the titration of PPV infectivity using serial dilutions of 1,000 times or more since the titer of spiked virus was high. Determination of the FFID₅₀ was performed using the fluorescence assay as described in the PPV FFID-infectivity assay of U.S. Patent Publication No. 2009/0226414.

The results are shown in Table 1.

TABLE 1
Inactivation of PPV using BPL conc 0.1-0.8% (v/v).
PPV was titered using a dilution method.

| | #1 | | #2 | | #3 | | #4 | |
|---|---|---|---|---|---|---|---|---|
| | Spiked PPV (FFID₅₀/g) titer | | | | | | | |
| | 5.20 × 10⁸ | | 5.20 × 10⁸ | | 5.20 × 10⁸ | | 5.20 × 10⁸ | |
| | | PPV | | PPV | | PPV | | PPV |
| | | Titer | | Titer | | Titer | Detected | Titer |
| | Detected | reduction | Detected | reduction | Detected | reduction | PPV Titer | reduction |
| BPL conc. | PPV Titer | (FFID₅₀/ | PPV Titer | (FFID₅₀/ | PPV Titer | (FFID₅₀/ | (FFID₅₀/ | (FFID₅₀/ |
| (v/v) | (FFID₅₀/g) | g) | (FFID₅₀/g) | g) | (FFID₅₀/g) | g) | g) | g) |
| 0% | 1.00 × 10⁸ | 0 | 5.62 × 10⁸ | 0 | 2.37 × 10⁸ | 0 | 1.33 × 10⁸ | |
| 0.1% | 4.22 × 10⁴ | 2.37 × 10³ | 4.22 × 10⁴ | 2.37 × 10³ | 5.62 × 10⁴ | 4.22 × 10³ | 0* | 1.33 × 10⁸ |
| 0.2% | 0* | >10⁵ | 0* | >10⁵ | 0* | >10⁵ | 0* | >10⁵ |
| 0.4% | 0* | >10⁵ | 0* | >10⁵ | 0* | >10⁵ | 0* | >10⁵ |
| 0.8% | 0* | >10⁵ | 0* | >10⁵ | 0* | >10⁵ | 0* | >10⁵ |

*Below the detection limit of 10³FFID₅₀/g

### Example 2

### Inactivation of EMCV Using BPL Conc. Between 0.1 0.8.%. (v/v) in PEP Preparations

Four different lots of pancrelipase powders were dissolved in 100 mM Tris, pH=8.3, at a concentration of 100 mg/ml. EMCV virus stock (prepared by Meng X J, Paul P S, Vaughn E M, Zimmerman J J. Development of a radiolabeled nucleic acid probe for the detection of encephalomyocarditis virus of swine. J Vet Diagn Invest. 1993 5:254-8) was mixed with the pancrelipase using slow shaking at room temperature for one day such that the final concentration of pancrelipase was 100 mg/ml. Aliquots were taken from the EMCV-spiked pancrelipase stocks and each was incubated with different concentrations of BPL (available from Sigma-Aldrich, St. Louis, Mo.) (except the control) for 48 hours at room temperature (20° C.). The level of inactivation was measured by the titration of EMCV infectivity on VERO cells using serial dilutions and determining the TCID₅₀ as described in Hierholzer, J. C., Killington, R. A., 1996. Virus isolation and quantitation. In: Mahy, B. W. J., Kangro, H. O. (Eds.), Virology methods manual. Academic Press, London, pp. 25-46.

The results are shown in Table 2.

TABLE 2
Inactivation of EMCV using BPL conc. between 0.1-0.8% (v/v)
EMCV was titered using a dilution method.

| | #1 | | #2 | | #3 | | #4 | |
|---|---|---|---|---|---|---|---|---|
| | Spiked EMCV (TCID₅₀/g) titer | | | | | | | |
| | 1.33 × 10⁸ | | 1.33 × 10⁸ | | 1.33 × 10⁸ | | 1.33 × 10⁸ | |
| | Detected | EMCV | Detected | EMCV | Detected | EMCV | Detected | |
| | EMCV | Titer | EMCV | Titer | EMCV | Titer | EMCV | EMCV |
| BPL | titer | reduction | titer | reduction | titer | reduction | titer | Titer |
| conc. | (TCID₅₀/ | (TCID₅₀/ | (TCID₅₀/ | (TCID₅₀/ | (TCID₅₀/ | (TCID₅₀/ | (TCID₅₀/ | reduction |
| (v/v) | g) | g) | g) | g) | g) | g) | g) | TCID₅₀/g) |
| 0.00% | 5.62 × 10⁷ | 0 | 5.62 × 10⁷ | 0 | 5.62 × 10⁷ | 0 | 1.00 × 10⁸ | 0 |
| 0.1.% | 4.22 × 10⁵ | 1.33 × 10² | 3.16 × 10⁵ | 1.78 × 10² | 0* | >10⁵ | 7.50 × 10⁴ | 1.33 × 10³ |
| 0.2.% | 0* | >10⁵ | 0* | >10⁵ | 0* | >10⁵ | 0* | >10⁵ |
| 0.4.% | 0* | >10⁵ | 0* | >10⁵ | 0* | >10⁵ | 0* | >10⁵ |
| 0.8.% | 0* | >10⁵ | 0* | >10⁵ | 0* | >10⁵ | 0* | >10⁵ |

*Below the detection limit of 10³TCID₅₀/g

### Example 3

### Inactivation of PPV by BPL with Conc. Between 0.04-0.25. % (v/v) in PEP Preparations

### The PPV was Titered Using the Chloroform/PEG Method

The experiment described in Example 1 was repeated except PEG-purified PPV was used for the infectivity assay as described in U.S. Patent Publication No. 2009/0226414.

The results are shown in Table 3. A somewhat higher residual infectivity was observed, possibly due to the increased sensitivity of the methods (less than 10 times initial dilution was used instead of 1,000 times dilution as in Example 1). Table 3 shows that 0.25% BPL inactivated the virus to about 0.02% of the initial infectivity (about 5,000-50,000 times decrease).

TABLE 3
Inactivation of PPV by BPL with conc. between 0.04-0.25%. PPV was titered using
chloroform/PEG method

| | #1 | | #2 | | #3 | | #4 | |
|---|---|---|---|---|---|---|---|---|
| | Spiked PPV (FFID₅₀/g) titer | | | | | | | |
| | 3.16 × 10⁶ | | 3.16 × 10⁶ | | 3.16 × 10⁶ | | 3.16 × 10⁶ | |
| | | PPV | | PPV | | PPV | | PPV |
| | Detected | Titer | Detected | Titer | Detected | Titer | Detected | Titer |
| BPL | PPV titer | reduction | PPV titer | reduction | PPV titer | reduction | PPV titer | reduction |
| conc. | (FFID₅₀/ | (FFID₅₀/ | (FFID₅₀/ | (FFID₅₀/ | (FFID₅₀/ | (FFID₅₀/ | (FFID₅₀/ | (FFID₅₀/ |
| (v/v) | g) | g) | g) | g) | g) | g) | g) | g) |
| 0.00% | 4.00 × | 0 | 3.00 × | 0 | 3.43 × | 0 | 2.00 × | 0 |
| | 10⁶ | | 10⁶ | | 10⁵ | | 10⁶ | |
| 0.04% | 6.40 × | 10 | 1.60 × | 10 | 1.13 × | 10 | 3.20 × | 10 |
| | 10⁵ | | 10⁵ | | 10⁵ | | 10⁵ | |
| 0.10% | 1.81 × | 10 | 2.26 × | 10² | 6.40 × | 10² | 1.13 × | 10² |
| | 10⁵ | | 10⁴ | | 10⁴ | | 10⁴ | |
| 0.25% | 7.07 × | 10⁵ | 7.07 × | 10⁵ | 7.07 × | 10⁵ | 1.41 × | 10⁴ |
| | 10¹ | | 10¹ | | 10¹ | | 10² | |

### Example 4

### Inactivation of Endogenous PPV by BPL Conc. 0.04 to 0.25% (v/v) in PEP

The experiment described in Example 1 was repeated except no additional PPV was added. Like Example 3, the infectivity assay was performed after PEG/chloroform purification.

The results are shown in Table 4. Table 4 shows that BPL inactivates the endogenous PPV of the PEP. In Example 3, 0.002% remaining infectivity could be measured (i.e., about 50,000 times inactivation at 0.25% BPL); this could not be achieved in Example 4 since not enough endogenous virus was present to measure the full scale of inactivation. Using the PPV FFID-infectivity assay described in U.S. Patent Publication No. 2009/0226414, 63 FFID₅₀/g PEP was the lowest level of virus that could be detected in this experiment.

TABLE 4
Inactivation of endogenous PPV by BPL with conc 0.04 vol. % to 0.25 vol. %.
PPV was titered using chloroform/PEG extraction method.

| | # 1 | | # 2 | | # 3 | | # 4 | |
|---|---|---|---|---|---|---|---|---|
| | Endogenous PPV (FFID₅₀/g) titer | | | | | | | |
| | 3.20 × 10³ | | 0.10 × 10³ | | 6.40 × 10³ | | 0.20 × 10³ | |
| | | PPV | | PPV | | PPV | | PPV |
| | Detected | Titer | Detected | Titer | Detected | Titer | Detected | Titer |
| | PPV titer | reduction | PPV titer | reduction | PPV titer | reduction | PPV titer | reduction |
| BPL conc. | (FFID₅₀/ | (FFID₅₀/ | (FFID₅₀/ | (FFID₅₀/ | (FFID₅₀/ | (FFID₅₀/ | (FFID₅₀/ | (FFID₅₀/ |
| (v/v) | g) | g) | g) | g) | g) | g) | g) | g) |
| 0.04% | 0.50 × 10² | 10 | 0* | 10³ | 4.00 × 10² | 10 | 0* | 10³ |
| 0.10% | 0* | 10³ | 0* | 10³ | 0.07 × 10² | 10³ | 0* | 10³ |
| 0.25% | 0* | 10³ | 0* | 10³ | 0* | 10³ | 0* | 10³ |

*Below the detection limit of 63 FFID₅₀/g PEP

### Example 5

### Inactivation of PPV Using BPL Conc. Between 0.004-0.4% (v/v) in a Higher Concentrations of PEP

### PPV was Titered Using a Dilution Method

The focus of this experiment was to measure the effect of increased PEP concentration on the efficiency of the inactivation procedure and to evaluate lower concentration ranges of BPL.

PPV virus stock (prepared using standard protocols (Arella et al., Physicochemical properties, production, and purification of parvoviruses. 1990, In Tijssen, P. (ed.), CRC handbook of parvoviruses. Boca Raton, Fla.: CRC Press, 11-30)) was diluted with water and this solution (containing 1.42×10¹⁰ FFID₅₀ PPV viruses) was used to suspend the 0.5 gram of PEP powder to yield a final concentration of 200 mg PEP/ml.

Tubes were incubated for one hour at 25° C. before the addition of BPL. BPL was added either directly to the PEP solution (for a final concentration of 0.4 vol. %) or it was first diluted ten and hundred times for final concentrations of 0.04 vol. % and 0.004 vol. %, respectively. After the addition of BPL, the solution was further mixed and incubated at 25° C. for a predetermined period (1, 2, 4, 6, or 8 hours). A sodium thiosulfate solution was freshly prepared on the day of each inactivation experiments. After the period expired for the BPL inactivation, sodium thiosulfate was added to the PEP solution, mixed intensively and incubated at least 6 hours at 4° C. to quench the excess BPL. After this step the infectivity titration experiments as described in U.S. Patent Publication No. 2009/0226414 were performed. All experiments were done using three parallel samples for each time point.

TABLE 5
Inactivation of PPV using BPL conc. between 0.004-0.4% (v/v).
PPV was titered using a dilution method.

| | 0.4% BPL | | 0.04% BPL | | 0.004% BPL | |
|---|---|---|---|---|---|---|
| | Detected | | Detected | | Detected | |
| | PPV titer | | PPV titer | | PPV titer | |
| | average | Log₁₀ of | average | Log₁₀ of | average | Log₁₀ of |
| Time (hr) | (FFID₅₀/g) | inactivation | (FFID₅₀/g) | inactivation | (FFID₅₀/g) | inactivation |
| 1 | 3.06 × 10⁷ | 2.8 | 4.01 × 10⁹ | 0.7 | 8.13 × 10⁹ | 0.4 |
| 2 | 8.69 × 10⁶ | 3.4 | 2.06 × 10⁹ | 1.0 | 5.96 × 10⁹ | 0.6 |
| 4 | 2.13 × 10⁶ | 4.0 | 1.54 × 10⁹ | 1.2 | 5.86 × 10⁹ | 0.58 |
| 6 | 3.26 × 10⁵ | 4.8 | 7.66 × 10⁸ | 1.5 | 6.65 × 10⁹ | 0.53 |
| 8 | 1.69 × 10⁵ | 5.1 | 4.91 × 10⁸ | 1.7 | 6.68 × 10⁹ | 0.53 |

### Example 6

### Inactivation of PPV by 0.8% (v/v) BPL at 4° C.

Samples prepared as in Example 1 were treated for four hours with 0.8% (v/v) BPL at 4° C.

TABLE 6
Inactivation of PPV by 0.8% (v/v) BPL at 4° C. PPV
was titered using the dilution method.

| | Sample infectivity expressed as | | | |
|---|---|---|---|---|
| | FFID₅₀/g log after treatment | | | |
| | with BPL for 4 h at 4° C. | | | |
| | | No BPL | 0.8% BPL | Log |
| Media | Control (0 h) | (4 h) | (4 h) | Reduction |
| Water | 10.2 | 10.4 | 5.0 | 5.2 |
| Mixture water + | 10.1 | 10.1 | 5.2 | 4.9 |
| IPA | | | | |

### Example 7

### Kinetics of PPV Inactivation by Treatment of PEP Samples with 0.4% BPL at 4° C.

Samples prepared in the same way as described in Example 1 were treated at 4° C. in an aqueous media (i.e. mixture of water and isopropyl alcohol). The PPV infectivity was determined by dilution method and, as illustrated in Table 7, a reduction of 2 logs can be obtained after 45 min of treatment.

TABLE 7
Average PPV Log reduction after treatment with 0.4% BPL at 4° C.
(PEP conc. 100 mg/mL)

| | Reaction Time | | | | |
|---|---|---|---|---|---|
| | 15 min | 30 min | 45 min | 60 min | 720 min |
| PPV titer | 1.1 | 1.6 | 2.0 | 2.2 | 4.8 |
| (log reduction) | | | | | |

### Examples 8-11

4.5 g pancrelipase (PEP) powder was dissolved in 44 ml ice cold 100 mM Tris (pH=8.3) by frequently mixing a 50-ml plastic tube that was kept on ice most of the time except when shaking. The dissolved sample was spiked with 1 ml (2×0.5 ml) PPV stock (prepared at the Institut National de la Recherche Scientifique, Québec, Canada using standard protocols (Arella et al., Physicichemical properties, production, and purification of parvoviruses. 1990, In Tijssen, P. (ed.), CRC handbook of parvoviruses. Boca Raton, Fla.: CRC Press, 11-30)) 10⁷ FFID₅₀/ml; see Example 8 and Table 8) and was mixed for another 10 minutes.

Three aliquots of the dissolved pancrelipase were transferred into tubes (A-C) and kept on ice while (A) no BPL—the control), (B) (0.4 vol % BPL), or (C) (0.8 vol % BPL) was added to them. After a brief mixing by a vortex machine, the tubes were incubated for 30 minutes at 25° C. At the end of the incubation, the tubes were transferred on to ice and immediately four aliquots were transferred from each experimental condition into new tubes (a-d). These tubes were placed into the mixture of acetone/dry ice for fast freezing. Later, the samples were stored at −80° C.

From the remaining solutions, aliquots from each experiment (using triplicates) were diluted 100 times in cell culture medium containing sodium thiosulfate and supplemented with traces of chloroform. Samples were briefly mixed and kept at 4° C. for at least 6 hours before further dilution for the FFID₅₀ infectivity determination. See Example 9.

The experiment above was repeated two more times (altogether: experiments 1-3).

The frozen samples were lyophilized at −45° C. for two days, and the pancrelipase powders were kept at 4-10° C. Samples a-b from all the experiments were dissolved in water. Infectivity measurements were done with diluted samples using the 400 times diluted samples as the lowest dilution step to determine the FFID₅₀. To easily compare the results for the different experiments, all of the obtained infectivity data were converted to specific infectivity (FFID₅₀/gram PEP). See Example 10 and Table 10.

The samples c-d from all the experiments were tested for lipase, protease, and amylase enzymatic activity using USP 31 monographed methods. See Example 11 and Table 11.

### Example 8

### Results

### Production and Titration of Stock PPV

Fresh virus stock was produced and titrated. Virus recovery from PEP samples was determined also after spiking. An equivalent PPV titer was recovered from these samples (Table 8).

TABLE 8
Infectivity of the PPV stock

| | Mean infectivity ± | Recovered PPV |
|---|---|---|
| Infectivity test | Standard Deviation | (FFID₅₀/mL in |
| (FFID₅₀/ml) | (FFIF₅₀/mL) | PEP) |
| 1.28 × 10⁷ | 9.35 ± 2.76 × 10⁶ | 2.079 × 10⁶ |
| 8.06 × 10⁶ | | |
| 1.01 × 10⁷ | | |
| 6.40 × 10⁶ | | |

### Example 9

### Results

### Inactivation of PPV by BPL and Subsequent Inactivation of BPL Using Sodium Thiosulfate

Table 9 presents the result of the inactivation of PPV by BPL for 30 min at 25° C. and subsequent inactivation of the BPL by incubating the BPL-containing PEP solutions with sodium thiosulfate for at least 6 hours at 4° C. As discussed above, three independent experiments with BPL inactivation after PPV spiking were followed by inactivation of BPL by sodium thiosulfate (3 parallels for each experiment).

TABLE 9
Inactivation of PPV by BPL subsequently Inactivated using Sodium Thiosulfate

| | | | | Mean | PPV titer |
|---|---|---|---|---|---|
| | | Results | Average Results | Infectivity ± | reduction |
| Treatment | Experiments | (FFID₅₀/g) | per Sample | SD | (FFID₅₀/g) |
| No BPL | A1 | 2.54 × 10⁶ | 1.60 × 10⁶ | 1.71 ± 0.29 × 10⁶ | 0 |
| | | 1.01 × 10⁶ | | | |
| | | 1.27 × 10⁶ | | | |
| | A2 | 2.01 × 10⁶ | 2.05 × 10⁶ | | |
| | | 2.54 × 10⁶ | | | |
| | | 1.60 × 10⁶ | | | |
| | A3 | 1.60 × 10⁶ | 1.49 × 10⁶ | | |
| | | 1.60 × 10⁶ | | | |
| | | 1.26 × 10⁶ | | | |
| 0.4% BPL | B1 | 2.50 × 10⁴ | 2.32 × 10⁴ | 2.63 ± 6.86 × 10⁴ | 0.65 × 10² |
| | | 2.50 × 10⁴ | | | |
| | | 1.98 × 10⁴ | | | |
| | B2 | 1.98 × 10⁴ | 2.15 × 10⁴ | | |
| | | 2.50 × 10⁴ | | | |
| | | 1.98 × 10⁴ | | | |
| | B3 | 3.14 × 10⁴ | 3.42 × 10⁴ | | |
| | | 3.96 × 10⁴ | | | |
| | | 3.14 × 10⁴ | | | |
| 0.8% BPL | C1 | 1.57 × 10⁴ | 1.05 × 10⁴ | 5.25 ± 5.25 × 10³ | 0.31 × 10⁴ |
| | | 0* | | | |
| | | 1.57 × 10⁴ | | | |
| | C2 | 0* | 0* | | |
| | | 0* | | | |
| | | 0* | | | |
| | C3 | 0* | 5.25 × 10³ | | |
| | | 1.57 × 10⁴ | | | |
| | | 0* | | | |

Note
0* means under detection limit

### Example 10

### Results

### Inactivation of PPV by BPL and Subsequent Freezing/Lyophilization of Samples to Inactivate the BPL

Inactivation results for PEP samples containing PPV inactivated by BPL at 25° C. for 30 min which was subsequently inactivated by freezing followed by lyophilization are presented in Table 10.

TABLE 10
Inactivation of PPV by BPL measured from Frozen and Lyophilized Samples

| | | PPV | | | | Reduction |
|---|---|---|---|---|---|---|
| | | infectivity | | | | of PPV titer |
| Treatment | Experiments | (FFID₅₀/g) | Average Results | | Mean ± SD | (FFID₅₀/g) |
| No BPL | A1a | 1.19 × 10⁶ | 1.74 × | 1.55 × | 1.55 ± | 0 |
| | | 2.28 × 10⁶ | 10⁶ | 10⁶ | 0.075 × | |
| | | | | | 10⁶ | |
| | A1b | 1.98 × 10⁶ | 1.37 × | | | |
| | | 0.75 × 10⁶ | 10⁶ | | | |
| | A2a | 1.61 × 10⁶ | 1.42 × | 1.47 × | | |
| | | 1.22 × 10⁶ | 10⁶ | 10⁶ | | |
| | A2b | 1.89 × 10⁶ | 1.53 × | | | |
| | | 1.16 × 10⁶ | 10⁶ | | | |
| | A3a | 0.71 × 10⁶ | 1.14 × | 1.62 × | | |
| | | 1.57 × 10⁶ | 10⁶ | 10⁶ | | |
| | A3b | 1.98 × 10⁶ | 2.10 × | | | |
| | | 2.22 × 10⁶ | 10⁶ | | | |
| 0.4% BPL | B1a | 0* | 0* | 5.74 × | 5.74 ± 5.74 × | 0.27 × 10³ |
| | | 0* | | 10³ | 10³ | |
| | B1b | 2.29 × 10⁴ | 11487 | | | |
| | | 0* | | | | |
| | B2a | 0* | 0* | 0* | | |
| | | 0* | | | | |
| | B2b | 0* | 0* | | | |
| | | 0* | | | | |
| | B3a | 2.29 × 10⁴ | 2.29 × | 1.14 × | | |
| | | 2.29 × 10⁴ | 10⁴ | 10⁴ | | |
| | B3b | 0* | 0* | | | |
| | | 0* | | | | |
| 0.8% BPL | C1a | 0* | 0* | 0* | 0* ± 0 | 1.55 × 10⁶ |
| | | 0* | | | | |
| | C1b | 0* | 0* | | | |
| | | 0* | | | | |
| | C2a | 0* | 0* | 0* | | |
| | | 0* | | | | |
| | C2b | 0* | 0* | | | |
| | | 0* | | | | |
| | C3a | 0* | 0* | 0* | | |
| | | 0* | | | | |
| | C3b | 0* | 0* | | | |
| | | 0* | | | | |

0* means under detection limit

### Example 11

### Results

### Enzymatic Activity after BPL Treatment and Freezing/Lyophilization of the Samples

Table 11 presents the lipase, protease, and amylase activity for aliquots of the pancrelipase after BPL treatment for 30 min at 25° C. followed by freezing and lyophilization (same treatment as for samples in Example 10).

TABLE 11
Enzymatic activity after BPL treatment, freezing, and lyophilization

| | Loss of Lipase | Loss of Protease | Loss of Amylase |
|---|---|---|---|
| Treatment | Activity | Activity | Activity |
| No BPL | N/A | N/A | N/A |
| 0.4% BPL | 19.79% | 0% | 11.40% |
| 0.8% BPL | 26.40% | 0% | 28.22% |

The precision of these results is between 5-10%.

### Discussion for Examples 8-11.

When the pancrelipase samples were tested immediately after PPV inactivation using BPL followed by treatment with sodium thiosulfate, the virus infectivity was reduced more than 60 times in the presence of 0.4 vol % BPL when incubated at room temperature for 30 minutes. Furthermore, 0.8% BPL inactivates PPV by three magnitudes of order under the same conditions. See Table 9.

As shown in Table 11, the lyophilized samples treated with 0.4% BPL and 0.8% BPL maintained the majority of lipase, protease, and amylase activity. These results demonstrated that PEPs can maintain high enzymatic activity after viral inactivation.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

From the foregoing description, one skilled in the art can easily ascertain the essential characteristics of this invention and, without departing from the spirit and scope thereof, can make various changes and modifications of the invention to adapt it to various usages and conditions.

1. A pancreatic enzyme preparation (PEP) having reduced viral infectivity comprising (i) one or more pancreatic enzymes and (ii) 3-hydroxypropionic acid, beta-propiolactone (BPL), or a mixture thereof.

2. The pancreatic enzyme preparation of claim 1 comprising (i) one or more pancreatic enzymes and (ii) 3-hydroxypropionic acid.

3. The pancreatic enzyme preparation of claim 1, wherein the preparation has been pre-treated with beta-propiolactone (BPL) and has a viral infectivity of porcine parvovirus (PPV) of at least 1 log below that of a preparation not treated with BPL.

4. The pancreatic enzyme preparation of claim 1, wherein the preparation has been pre-treated with beta-propiolactone (BPL) and has a viral infectivity of non-enveloped viruses of at least 1 log below that of a preparation not treated with BPL.

5. The pancreatic enzyme preparation of claim 1, wherein the preparation has been pre-treated with beta-propiolactone (BPL) and has a viral infectivity of enveloped viruses of at least 1 log below that of a preparation not treated with BPL.

6. The pancreatic enzyme preparation of claim 1, wherein the preparation has been pre-treated with beta-propiolactone (BPL) and has a viral infectivity of porcine encephalomyocarditis virus (EMCV) of at least 1 log below that of a preparation not treated with BPL.

7. The pancreatic enzyme preparation of claim 1, wherein the preparation has a porcine parvovirus (PPV) viral infectivity of less than about 10³ FFID₅₀/g PEP.

8. The pancreatic enzyme preparation of claim 1, wherein at least one pancreatic enzyme in the preparation is derived from an animal source.

9. The pancreatic enzyme preparation of claim 1, wherein one or more enzymes is selected from lipases, proteases, and amylases.

10. The pancreatic enzyme preparation of claim 1, wherein the preparation comprises pancrelipase.

11. A pharmaceutical composition having reduced viral infectivity comprising the preparation of claim 1, and optionally a pharmaceutically acceptable excipient.

12. A solid oral dosage form comprising
a pancreatic enzyme preparation (PEP) having reduced viral infectivity, wherein (i) the PEP comprises from about 1,000 to about 60,000 USP units lipases, from about 3,000 to about 360,000 USP units proteases, and from about 3,000 to about 360,000 USP units amylases, and (ii) the PEP has been pre-treated with beta-propiolactone (BPL) and has a viral infectivity of porcine parvovirus (PPV) of at least 1 log below that of a PEP not treated with BPL, and
optionally, one or more pharmaceutically acceptable excipients.

13. The solid oral dosage form of claim 12, wherein the dosage form is in form of a powder, a tablet, a mini-tablet, microspheres, a prill, or a capsule.

14. A solid oral dosage form comprising
a pancreatic enzyme preparation (PEP) having reduced viral infectivity, wherein (i) the PEP comprises from about 1,000 to about 60,000 USP units lipases, from about 3,000 to about 360,000 USP units proteases, and from about 3,000 to about 360,000 USP units amylases, and (ii) the PEP has a PPV infectivity below about 100 FFID₅₀/g PEP, and
optionally, one or more pharmaceutically acceptable excipients.

15-32. (canceled)

33. A method for treating pancreatic insufficiency in a patient in need thereof comprising administering to the patient a therapeutically effective amount of the pharmaceutical composition of claim 11.

34. A method of preparing a pancreatic enzyme preparation (PEP) comprising the step of (a) reacting beta-propiolactone (BPL) with a preparation containing one or more pancreatic enzymes for a sufficient time to reduce a viral infectivity in the preparation.

35. The method of claim 34, wherein the viral infectivity of non-enveloped viruses in the preparation after reaction with BPL is at least 1 log lower compared to the viral infectivity of non-enveloped viruses of the preparation prior to reaction with BPL.

36. The method of claim 34, wherein step (a) comprises:
(i) adding BPL to a solution or suspension containing the preparation of one or more pancreatic enzymes, and
(ii) incubating BPL in the solution of step (i) for a time sufficient to decrease the viral infectivity in the solution.

37. The method of claim 36, wherein step (i) comprises adding to a solution or suspension comprising from about 100 to about 200 mg PEP/ml, from about 0.004% to about 1.0% (v/v) of BPL.

38. The method of claim 34, wherein the reaction is carried out for about 30 minutes to about 72 hours.
